(19) United States
(12) Patent Application Publication
(10) Pub. No.: US 2024/0091519 A1
(43) Pub. Date: Mar. 21, 2024

(54) LEAKAGE CONNECTOR FOR A DIALYSIS MACHINE, A LEAKAGE CONSTRUCTION EQUIPPED WITH IT AS WELL AS A DIALYSIS MACHINE

(71) Applicant: B. Braun Avitum AG, Melsungen (DE)

(72) Inventors: Marco Jakobi, Uder (DE); Oliver Schaefer, Neuenstein (DE); Thorsten Thomas, Schwalmstadt (DE)

(21) Appl. No.: 18/465,346

(22) Filed: Sep. 12, 2023

(30) Foreign Application Priority Data
Sep. 15, 2022 (DE) 10 2022 123 642.0

(51) Int. Cl. A61M 39/10; A61M 1/16

(52) U.S. Cl. CPC A61M 39/10; A61M 1/1601

(57) ABSTRACT

A dialysis machine includes a leakage construction having a leakage collecting device and a leakage connector. The leakage connector is preferably a leakage adapter arranged between an opening of the leakage collecting device and a hose. The leakage connector has a main body to which a seal is attached, which can be brought into sealing contact with the leakage collecting device.

### CROSS-REFERENCE TO RELATED APPLICATION(S)

This application claims priority under 35 U.S.C. § 119 to German Application No. 10 2022 123 642.0, filed on Sep. 15, 2022, the content of which is incorporated by reference herein in its entirety.

### FIELD

The present disclosure relates to a leakage connector for a or of a dialysis machine, which is configured to drain leakage fluid, in particular from a filter box for the or of the dialysis machine. The present disclosure further relates to a leakage construction for the or of the dialysis machine comprising such a leakage connector, and the dialysis machine comprising the leakage construction.

### BACKGROUND

In some generally known dialysis methods, such as hemodialysis, dialysis machines use a dialysis fluid which is fed to a dialysis filter in order to remove certain blood components from a patient's blood flowing through the dialysis filter (dialyzer) according to the diffusion principle, among other principles. In order to avoid impurities, the fresh dialysis fluid is filtered via at least one dialysis fluid filter before it is fed to the dialysis filter. This dialysis fluid filter is usually a hollow fiber filter and is used to produce ultra-pure dialysis fluid for dialysis treatments.

The technical background of the dialysis fluid filter is that even if the dialysis machine has been properly cleaned and disinfected, the permeate necessary to produce fresh dialysis fluid in the dialysis machine and the bicarbonate concentrate can be sources of possible contamination of the dialysis fluid produced. Such impurities are filtered out by the dialysis fluid filter.

Dialysis fluid filters are usually located at the rear of the dialysis machine, away from the operator, and are housed in a special/separate filter box within the housing of the dialysis machine. Such filter boxes normally also have a maintenance access that opens to the rear of the dialysis machine and can be closed via a rear door. The filter box is also separated from the rest of the interior of the machine housing by a rear wall (opposite the door), to which holders for receiving the dialysis fluid filters (usually in cartridge form) are attached. In addition, inlet and outlet lines lead into the filter box, through which the dialysis fluid filters can be interposed in the fluid circuit of the fresh dialysis fluid. The inlet and outlet lines also have connections for detachable connection to the dialysis fluid filters.

In general, leakage is to be expected in fluid-carrying lines, especially in the region of connections, possibly as a result of faulty assembly, defective seals or simply as a result of holes/cracks in the lines themselves. Since dialysis machines also have so-called balancing devices, with the help of which the machine electronics can detect the amount of fresh and used dialysis fluid and, if applicable, can calculate from this the amount of fluid that has been withdrawn from a patient, there is a need to detect and locate leakages as early as possible in order to avoid measurement and/or calculation errors, for example in the balancing device.

EP 2 219 705 B1 discloses a machine for extracorporeal blood treatment, in the lower region of which an accumulation or collection container for leakage fluid of any kind is arranged, which is then conducted via a hose to a leakage detector. Therefore, the hose has to be connected to the accumulation container, for which a sealing leakage connector has to be provided. This is complex and costly. The correct installation of a seal between the accumulation container and the hose can be difficult with such a leakage connector. In the machine shown, this applies in particular because the targeted accumulation of leakage fluid in the accumulation container results in the seal being permanently exposed to leakage fluid. Moreover, with such an arrangement of an accumulation container, it is possible to determine the amount of leakage, but not the exact location of the leakage.

### SUMMARY

The object of the present disclosure is to provide a leakage connector which is configured to discharge leakage or flushing fluid from an opening of a leakage collecting device of a dialysis machine, and furthermore to simplify a leakage construction of a dialysis machine with such a leakage connector and finally also to simplify a dialysis machine with regard to the assembly of the leakage connector. The leakage connector is to be reliably sealed with respect to the leakage collecting device.

In the following, instead of the term leakage fluid, the term leakage is used for simplification. Leakage thus means leakage fluid in the present writing. Furthermore, the leakage collecting device is preferably a component of a filter box receiving a number of dialysis fluid filters, the dialysis fluid filters preferably being mountable on a filter box rear wall or a filter box access door.

The basic idea of the present disclosure is in principle to provide a leakage connector in the form of a connection adapter as an independent, separate component which, according to the general operating principle of an adapter, has or comprises an outlet connection specially configured for fluid coupling to a hose or a line and a universal inlet connection connectable to some kind of through-opening on the side of a leakage-fluid collection device, so that a hose or a line can be conveniently and safely connected to the through-opening of the leakage-fluid collection device configured in some way. For this purpose, the connection adapter has at least
a hose connector or sleeve, the shape of which is adapted to a hose or pipe,
a connection mask in the form of a mounting/contact plate having a contact surface, in which an aperture forming an inlet is formed, to which the sleeve is fluid-connected, and
a sealing ring or sealing bead, which is attached or applied to the contact surface and surrounds the aperture at a certain radial distance.

The mounting/contact plate thus serves as a geometrically simple connection flange that only has to be pressed against a wall containing the passage opening of the leakage-fluid collection device, for example via screws, rivets or similar connecting means without having to take the shape of the passage opening into account.

The leakage connector, provided as a separate mounting component, is thus configured to drain leakage or also flushing liquid from an opening of a leakage collecting device of a dialysis machine. The leakage collecting device may be formed by a tray or a trough or a funnel or a container or the bottom of a separate compartment such as a filter box. The leakage collecting device does not have a collecting area in which leakage or rinsing liquid is accumulated. The opening is arranged e.g. at a rear wall of the compartment, for example of the filter box. The mountable leakage connector has a frame-type main body with a mounting/contact plate having or forming a leakage inlet and a leakage outlet, the leakage inlet and the leakage outlet being fluidically connected to each other via a channel (line) formed in or on the main body. Thus, the leakage connector is configured to transfer the leakage or flushing fluid collected in the leakage collecting device. The leakage connector has a circumferential seal/sealing ring/sealing bead which is fastened (fixed) to the main body and in particular to the mounting/contact plate, i.e. which (completely) surrounds the leakage inlet, and is intended to be pressed on/placed against a surface/wall of the leakage collecting device. This simplifies the assembly of the leakage connector on the leakage collecting device concerned, because the seal/sealing ring/sealing bead does not have to be fitted into an opening on the leakage collecting device and loss of the seal is still prevented. Furthermore, the sealing of the leakage connector with respect to the leakage collecting device is optimized, since slipping of the seal during assembly is prevented and thus a correct fit of the seal around the outlet opening of the leakage collecting device or around the leakage inlet of the leakage connector (leakage connector element) is ensured.

It is particularly preferred if the seal is a PU foam seal that is molded onto the main body. This further simplifies the assembly of the leakage connector on the leakage collecting device and further optimizes the secure sealing of the leakage connector against the leakage collecting device.

In a preferred concrete embodiment, the leakage outlet is formed with a sleeve which is preferably formed integrally with the main body and which can also be referred to as a nozzle. The sleeve is connected at one end to the leakage inlet and can preferably have an angulation, in particular a right-angled angulation, in its course/extension, so that its free end portion extends at right angles to the leakage inlet. A hose can be pushed onto the sleeve or its free end portion. The leakage outlet (separate leakage outlet element) is thus configured as a leakage adapter, so to speak, via which the leakage outlet opening of the leakage collecting device can be connected to the hose. The shape and dimensions of the leakage outlet opening of the leakage collecting device can thus be almost any desired ones, as long as it is located entirely within the area surrounded by the seal of the leakage outlet.

Preferably, the sleeve is stabilized by at least one reinforcing portion or reinforcement land/rib of the main body, which surrounds the sleeve circumferentially. The rib is aligned at a distance, preferably at a parallel distance, from the mounting/contact plate and is joined to it in one piece by a base plate. In addition, the rib is bent at its opposite rib ends and led back to the mounting/contact plate, whereby the rib together with the mounting/contact plate forms a kind of box/frame structure which is closed on an underside by the base plate and is open on an upper side. Base plate and mounting/contact plate thus form a kind of angle piece which is stiffened by the at least one rib.

If the sleeve has a—preferably circumferential—hose seal formed integrally on the sleeve, the hose can be reliably sealed off from the sleeve.

In a particularly preferred further development of the leakage connector, the circumferential seal of the leakage inlet is partially or completely surrounded by a contact surface, which is formed by the mounting/contact plate. This ensures a correct position of the main portion relative to the leakage collecting device concerned, if this has a corresponding outer contact surface. This is the case, for example, when the leakage collecting device is formed by the filter box and has the rear wall in which the opening is located. As a result, the seal is evenly applied to the opening of the leakage collecting device. This further optimizes the sealing of the leakage connector against the leakage collecting device.

The contact surface can be formed on the contact plate as described above. Preferably, the contact plate is stabilized with at least one reinforcing portion or reinforcement crosspiece of the main body. The sleeve may be configured in such a way that its angulation with respect to the mounting/contact plate is arranged behind the last rib, so that the free end portion of the sleeve is guided along the rib virtually outside the box formed by the rib, the base plate and the mounting/contact plate.

When the leakage connector is mounted, the contact surface is preferably approximately vertical. Then the channel has a slope. Then the leakage can flow through the leakage connector due to gravity without leakage collecting in it.

If the channel has the at least one bending or angulation, the sleeve may be oriented approximately parallel to the contact surface or approximately parallel to a plane defined by the contact surface. This can save installation space, or more precisely, depth of the dialysis machine concerned. This applies in particular if the hose, which is pushed onto the sleeve, is curved downwards in a bend-free manner.

In one embodiment, the sleeve may also be angled ‘downward’ to allow fluid to flow smoothly into a hose connected to the sleeve.

The leakage construction according to the present disclosure has the stated leakage collecting device. The leakage collecting device has the stated opening, to which the leakage inlet of the leakage connector described above is attached, e.g. clamped, in a sealing manner via a retaining device. Thus, the advantages already mentioned above are achieved, i.e. the assembly of the leakage connector to the leakage collecting device is simplified, since a loss of the seal is prevented. Furthermore, the sealing of the leakage connector with respect to the leakage collecting device is optimized, since slipping of the seal during assembly is prevented and thus a correct fit of the seal is ensured.

When the leakage collecting device is formed by the filter box of the dialysis machine, the leakage can be associated with the dialysis fluid filter contained therein, including the lines and seals therein.

In a preferred embodiment, the leakage collecting device has a land that is sectionally inserted between a first land contact and a second land contact of the main body of the leakage connector. In an operating position of the affected dialysis machine, the land is approximately horizontal, and the first land contact is an upper land contact, while the second land contact is a lower land contact.

When the leakage collecting device is formed by the filter box, it is preferred for manufacturing reasons that the land is flat and formed in one piece with a substantially flat bottom or bottom portion of the filter box. The bottom portion may be raised relative to a further bottom portion of the filter box, resulting in a stepped bottom.

Preferably, the land has a recess into which the main body is inserted in portions. The recess serves as a positioning aid and is arranged in such a way that the leakage inlet and thus its seal are positioned to match the opening in the assembled state.

In the case of the recess serving as a positioning aid, the first and second land contact of the main body may each consist of three portions angled toward each other.

In the case of the recess serving as a positioning aid, this and/or the main body preferably has at least one insertion chamfer. Preferably, the recess and the main body each have a pair of mirror-symmetrical insertion chamfers on both sides. During assembly of the leakage connector, these serve as insertion aids in the plane of the land, i.e. in the operating position in the horizontal direction.

The retaining device preferably has screws that extend through apertures of the land and are screwed into screw holes of the main body. The screw holes may be blind holes. Alternatively, the retaining device may be a latching mechanism.

Insertion chamfers may also be provided perpendicular to the plane of the land, i.e. in the operating position of the dialysis machine in the vertical direction. These are formed, for example, at the screw holes of the main portion.

The opening, the leakage inlet, the channel, the sleeve and the hose may be shaped and dimensioned in such a way that a flow of up to 1200 ml/min is possible due to gravity alone. This means that the leakage construction may also be used for flushing.

The dialysis machine according to the present disclosure comprises the aforementioned leakage collecting device formed, for example, by the filter box of the dialysis machine. The leakage collecting device comprises the stated opening, to which the leakage inlet of the leakage connector described above is attached, e.g. clamped, in a sealing manner via a retaining device. Thus, also with regard to the dialysis machine, the advantages already mentioned above are achieved, i.e. the assembly of the leakage connector to the leakage collecting device is simplified, since a loss of the seal is prevented. Furthermore, the sealing of the leakage connector with respect to the leakage collecting device is optimized, since slipping of the seal during assembly is prevented and thus a correct fit of the seal is ensured.

In the prior art according to the publication EP 2 219 705 B1, the leakage is not directly forwarded to the leakage detector, but is collected beforehand. More specifically, the disclosed machine has a support structure with a chamber in which the leakage is collected and conveyed via a drain channel to a discharge region. The drainage area is located outside in the base region of the device where the leakage is detected by the leakage detector.

In the operating position of the dialysis machine according to the present disclosure, the opening is preferably arranged at the lowest point of the leakage collecting device and the channel preferably has a slope so that the sleeve is arranged below the leakage inlet. Below the sleeve, e.g. in a base region of the dialysis machine, a leakage detector is arranged. The leakage detector is connected to the sleeve via a monotonically descending hose, or the leakage detector is arranged below an exit of the monotonically descending hose. The term “monotonically descending”, as used herein when referring to a hose or channel, means that the hose or channel only pitches downwardly from its inlet end to prevent collection of liquid in the hose or channel. The exit of the hose and the leakage detector may be connected directly or indirectly. The hose may extend into a detection region where the leakage detector is located, or the hose may meet the base at any point, from where the leakage fluid can flow specifically to the leakage detector. In this case, the leakage can flow through the leakage adapter and the hose due to gravity without leakage being able to collect in it. Furthermore, a collection point for leakage is not provided in the detection region of the leakage detector. This enables immediate and instantaneous detection of the leakage and a corresponding message or other consequence, in particular a stop of the flow through the dialysis fluid filter arranged in the filter box.

In a particularly preferred further development, the leakage detector is configured in such a way that it responds after 5-7 seconds at a volume of 100 ml and the dialysis machine stops the flow through the dialysis fluid filter arranged in the filter box.

### BRIEF DESCRIPTION OF THE DRAWINGS

FIG. 1 shows a part of a dialysis machine in a perspective rear view with a filter box;

FIG. 2 shows a part of the filter box from FIG. 1 in a perspective view from the inside;

FIG. 3 shows a leakage construction according to an embodiment;

FIG. 4 shows the leakage adapter from FIG. 3 in a perspective view;

FIG. 5 shows the leakage adapter from FIG. 3 in a further perspective view;

FIG. 6 shows a view of the leakage adapter from FIG. 3;

FIG. 7 shows a portion of the leakage adapter from FIG. 3 in a further view;

FIG. 8 shows a portion of the back of the filter box with the leakage adapter of FIG. 2 in a bottom view; and

FIG. 9 shows a portion of the rear wall with the leakage adapter from FIG. 3 in a cutaway view.

### DETAILED DESCRIPTION

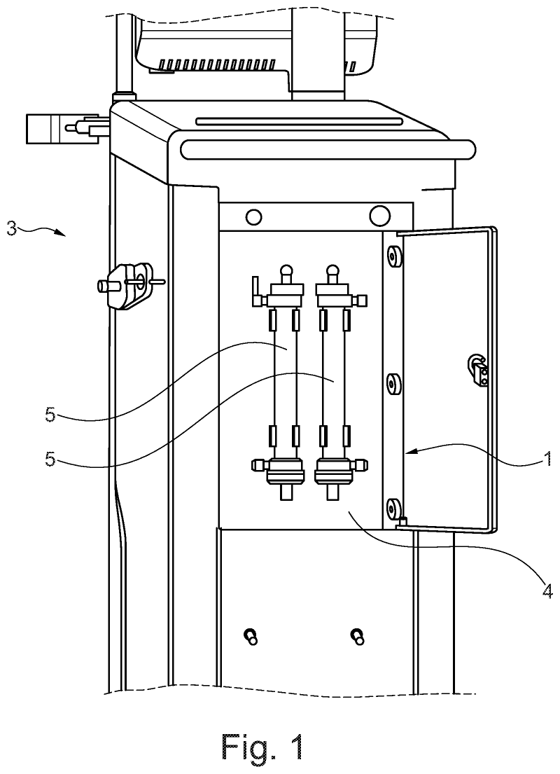

FIG. 1 shows a part of a dialysis machine 3 in a perspective view from the rear. A filter box 1 is shown at the rear with its rear door open. This provides a view of two dialysis fluid filters 5, which are used to generate ultra-pure dialysis fluid for dialysis treatments.

Furthermore, a separation wall can be seen, which is arranged in a central region in relation to the entire dialysis machine 3. In relation to the filter box 1, the separation wall is at the rear and is therefore referred to as rear wall 4.

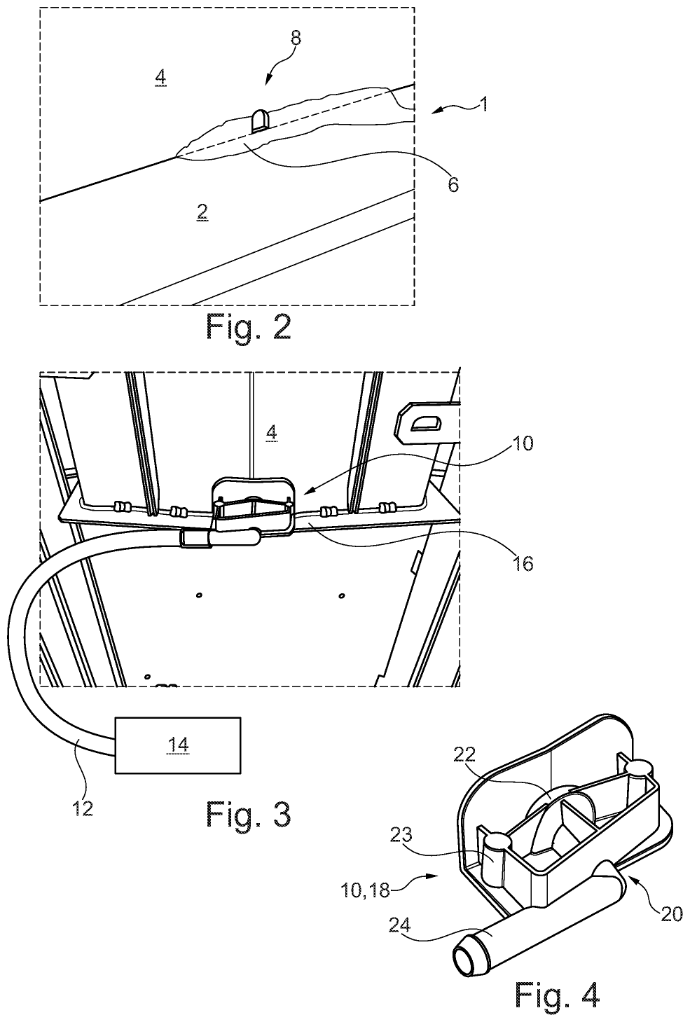

FIG. 2 shows a part of the filter box 1 of the dialysis machine 3 of FIG. 1 in a perspective view from the inside. A plate-shaped bottom portion or bottom 2 and the plate-shaped rear wall 4 are provided. A corner portion formed between the bottom 2 and the rear wall 4 is supplied with leakage 6 in larger unrealistic amounts for demonstration purposes. This immediately flows through an opening 8 to a rear region of the filter box 1.

FIG. 3 shows the leakage construction according to an example. A part of the rear wall 4 of the filter box 1 of the dialysis machine 3 from FIG. 2 is shown with a leakage connector configured as a separate leakage adapter 10. The leakage adapter 10 is attached to the opening 8 shown in FIG. 2 and directs the leakage 6 to a hose 12 which may be made of silicone, for example. The monotonically descending hose 12 guides the leakage 6 to a leakage detector 14, which can be arranged below the opening 8, e.g. in a base region of the dialysis machine 3 (shown in FIG. 1). Due to gravity, the leakage 6 can flow through the leakage adapter 10 and the hose 12 without leakage 6 being able to collect therein. Furthermore, no collection point for leakage 6 is provided in the detection region of the leakage detector 14 either. This allows immediate and prompt detection of the leakage, so that the user can react and eliminate the leakage, for example, by stopping the flow through the dialysis fluid filter 5 arranged in the filter box 1.

The leakage adapter 10 is attached to a dialysis machine-side land 16 which, in the operating position of the dialysis machine 3, extends horizontally or approximately horizontally from rear wall 4 (with respect to filter box 1) to the rear. The land 16 is formed integrally with the bottom 2 shown in FIG. 2. The rear wall 4 and the bottom 2 together with the land 16 are made of metal.

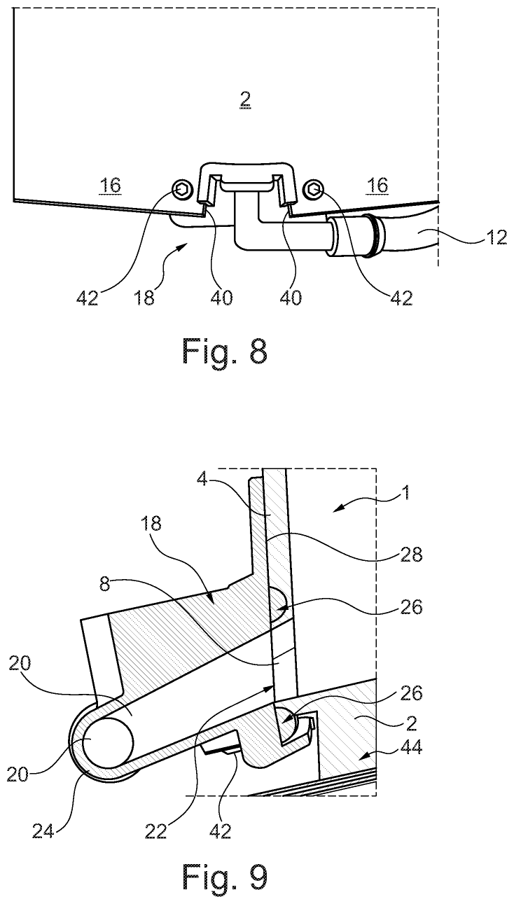

This attachment of the leakage adapter 10 to the land 16 will be discussed in more detail with reference to FIGS. 8 and 9.

FIG. 4 shows the leakage adapter 10 from FIG. 3 in a perspective view. It has a main body 18 made of plastic, preferably by injection molding, in which an angled channel 20 is formed for the leakage 6. The channel 20 connects a leakage inlet 22 to a leakage outlet formed as a sleeve 24 or spigot. The channel 20 is also monotonically descending, so that the leakage 6 cannot collect in it either.

Two circular cylindrical portions 23 of the main body 18 can also be seen, in which screw holes (not visible in FIG. 4) are formed as blind holes. The circular cylindrical portions 23 are connected to the leakage inlet 22 via a respective reinforcing portion.

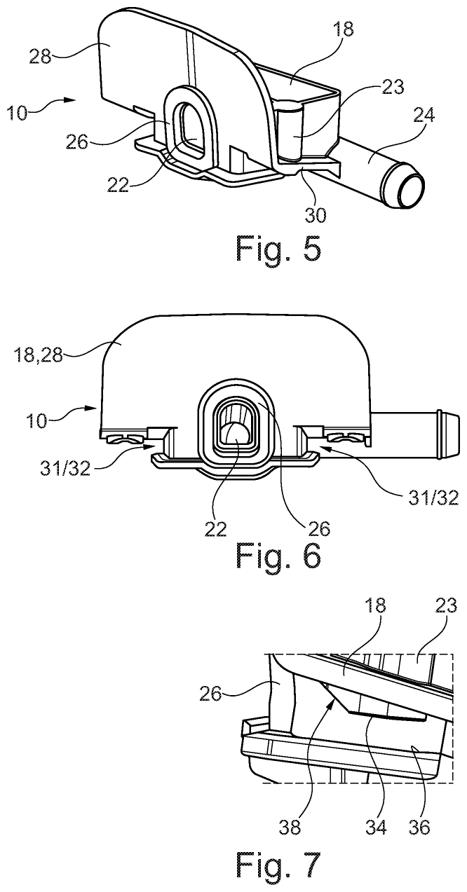

FIG. 5 shows the leakage adapter 10 from FIG. 3 in a further perspective view. In particular, a seal 26 can be seen surrounding the leakage inlet 22. The seal 26 is a PU foam seal molded onto the main body 18. The PU foam seal is largely surrounded by a contact surface 28, which is formed on a contact plate that is formed integrally with the main body 18.

In the assembled state of the leakage adapter 10 (shown in FIG. 3), the seal 26 of the leakage adapter 10 surrounds the opening 8 of the rear wall 4 in a sealing manner. A uniform contact of the seal 26 with the rear side of the rear wall 4 is ensured by the contact surface 28 of the main body 18, which is in comparatively large contact with the rear side of the rear wall 4.

The sleeve 24 is connected to the adjacent circular cylindrical portion 23 via a reinforcing portion 30.

FIG. 6 shows a further view of the leakage adapter 10 from FIG. 3. It can be seen that free spaces 31 or slots are provided below the contact surface 28. The land 16 shown in FIG. 3 is received in these spaces during assembly of the leakage adapter 10. Furthermore, insertion chamfers 32 are provided on the main body 18 on both sides of the seal 26, which act in a level or horizontal direction during assembly and result in the seal 26 being positioned exactly in front of the opening 8 on the rear side of the rear wall 4.

FIG. 7 shows a portion of the leakage adapter 10 from FIG. 3 in a further view. A first (upper) land contact 34 and a second (lower) land contact 36 can be seen, between which the land 16 shown in FIG. 3 is received in portions during assembly of the leakage adapter 10.

An insertion chamfer 38 is formed at each of the two mouths of the screw holes formed in the circular cylindrical portions 23, of which only one insertion chamfer 38 is shown in FIG. 7. The insertion chamfers 38 act in a vertical direction.

FIG. 8 shows the bottom 2 with the fully assembled leakage adapter 10 from FIGS. 3 to 7 in a view from below. In this view, the bottom 2 of filter box 1 is shown in the upper part of the figure and the land 16, which is formed integrally therewith, is shown in the lower part of the figure. It can be seen that the land 16 has a recess with insertion chamfers 40 into which the main body 18 of the leakage adapter 10 is inserted in portions. Then, in the embodiment shown, the main body 18 and thus the leakage adapter 10 are secured via two screws 42.

FIG. 9 shows a portion of the rear wall 4 with the leakage adapter 10 mounted as shown in FIG. 8. In typical CAD fashion, the seal 26 running around the opening 8 is shown as if it had penetrated the flat rear side of the rear wall 4. The angulation of the channel 20 can be seen. This minimizes the space required for the leakage arrangement, since the hose 12 (not shown in FIG. 9) is attached without kinks in a downward arc.

Below the bottom 2 of the filter box 1, a part of a power-adaptor carrier 44 of the dialysis machine 3 is still shown.

The leakage construction with the opening 8 of the filter box 1 and with the leakage inlet 22 and with the channel 20 and with the sleeve 24 and with the hose 12 are shaped and dimensioned in such a way that a flow of up to 1200 ml/min is possible due to the gravity of the leakage 6 or the flushing agent alone. This means that the leakage construction is also suitable for rinsing the filter box.

The bottom 4 and the opening 8 and the leakage inlet 22 and the channel 20 and the sleeve 24 and the hose 12 and the leakage detector 14 are arranged one above the other in such a way that the gravity of the leakage 6 or of the rinsing agent alone achieves its discharge without any appreciable liquid residues being able to accumulate anywhere along this path.

1. A leakage connector comprising a leakage connector adapter formed and configured for attachment to an opening of a leakage collecting device of a dialysis machine, the leakage connector having a main body with a leakage inlet and a leakage outlet interconnected with the leakage inlet via a channel formed in or on the main body, the leakage inlet being formed in a mounting/contact plate forming a contact surface and comprising a seal that is fixed to the main body and on the contact surface and surrounds the leakage inlet.

2. The leakage connector according to claim 1, wherein the seal is a PU foam seal that is molded onto the main body.

3. The leakage connector according to claim 1, wherein the leakage outlet is formed by or on a sleeve that is formed integrally with the main body.

4. The leakage connector according to claim 1, wherein the seal is partially or completely surrounded by the contact surface.

5. The leakage connector according to claim 4, wherein the channel is inclined or slanted with respect to the contact surface.

6. The leakage connector according to claim 1, wherein the channel comprises a bending or angulation.

7. A leakage construction for or of a dialysis machine comprising:
a leakage connector according to claim 1; and
a leakage collecting device comprising an opening,
the leakage connector being attached to the opening in a sealing manner via a retaining device.

8. The leakage construction according to claim 7, wherein the leakage collecting device comprises a land having an edge inserted between a first land contact and a second land contact of the main body.

9. The leakage construction according to claim 8, wherein the land has a recess into which the main body is inserted in portions.

10. The leakage construction according to claim 9, wherein the recess and/or the main body has/have at least one insertion chamfer.

11. The leakage construction according to claim 8, wherein the retaining device comprises screws extending through apertures of the land and screwed into the main body.

12. The leakage construction according to claim 8, wherein the leakage collecting device is formed by a filter box of the dialysis machine, and wherein the land is flat and integrally formed with a bottom or bottom portion of the filter box.

13. A dialysis machine comprising:
a leakage construction having:
a leakage connector according to claim 1; and
a leakage collecting device comprising an opening,
the leakage connector being attached to the opening in a sealing manner via a retaining device; and
a leakage detector connected to the leakage construction.

14. The dialysis machine according to claim 13, wherein:
the opening is arranged at a lowest point of the leakage collecting device,
the leakage outlet of the leakage adapter is arranged below the leakage inlet of the leakage adapter,
the leakage detector is arranged below the leakage outlet, and
the leakage detector is connected to the leakage adapter via a monotonically descending hose.

15. The dialysis machine according to claim 13, wherein:
the opening is arranged at a lowest point of the leakage collecting device,
the leakage outlet of the leakage adapter is arranged below the leakage inlet of the leakage adapter,
the leakage detector is arranged below the leakage outlet, and
the leakage detector is arranged below an exit of a monotonically descending hose.
